(19) United States
(12) Patent Application Publication
(10) Pub. No.: US 2012/0095219 A1
(43) Pub. Date: Apr. 19, 2012

(54) PROCESS FOR PREPARING BRINZOLAMIDE

(75) Inventors: Arul Ramakrishnan, Hyderabad (IN); Anil Kumar Soni, Hyderabad (IN); Sujit Das Adhikari, Kolkotta (IN); Kommula Srinivasa Rao, East Godavari (IN); Soumendu Paul, Hyderabad (IN); Ganta Srinivasulu, Hyderabad (IN)

(73) Assignee: Azad Pharmaceutical Ingredients AG, Schaffhausen (CH)

(21) Appl. No.: 13/256,317

(22) PCT Filed: Mar. 12, 2010
(86) PCT No.: PCT/EP10/53216

§ 371 Date: Dec. 5, 2011

Related U.S. Application Data
(60) Provisional application No. 61/159,898, filed on Mar. 13, 2009.

(51) Int. Cl. C07D 513/04; C07D 409/04; C07D 333/34

(52) U.S. Cl. 544/48; 549/64; 549/60; 549/65

(57) ABSTRACT

The present invention refers to the preparation and purification of brinzolamide as well as to novel compounds useful in such processes.

The present invention refers to the preparation and purification of brinzolamide as well as to novel compounds useful in such processes.

Brinzolamide [(4R)-4-(ethylamino)-3,4-dihydro-2-(3-methoxypropyl)-2H-thieno[3,2-e]-1,2-thiazine-6-sulfonamide-1,1-dioxide] is a carbonic anhydrase inhibitor useful for the treatment of glaucoma. The product and its manufacture is described e.g. in U.S. Pat. No. 5,240,923, U.S. Pat. No. 5,378,703 and U.S. Pat. No. 5,461,081.

The present inventors have identified novel processes for preparing and purifying brinzolamide. Further, the present inventors have identified novel compounds useful in such processes.

A first aspect of the present invention refers to a process for preparing brinzolamide (VIII) comprising the steps:
(a) reacting compound (I)

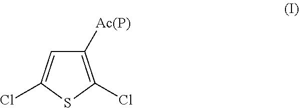

wherein Ac(P) is an acetyl group or a masked, e.g. protected or reduced acetyl group, with a sulphonating reagent, particularly a sulphite salt, to obtain compound (II)

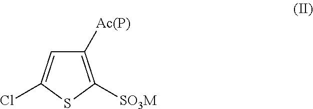

wherein Ac(P) is as described above and M is a cation, particularly Na⁺,
(b) reacting compound (II) with 3-methoxy amino propane to obtain compound (Ill)

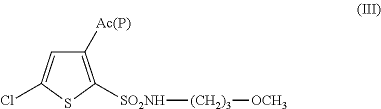

wherein Ac(P) is as described above,
(c) optionally masking the acetyl group of compound (Ill) with an acetyl protecting agent, particularly ethylene glycol, or a reducing agent, to obtain compound (IV)

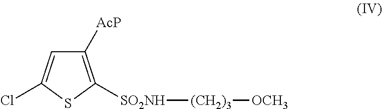

wherein AcP is a masked, e.g. protected or reduced acetyl group, particularly

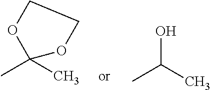

(d) (i) reacting compound (IV) with an organo metal (organometallic) compound followed by treatment with SO₂ and subsequently with hydroxylamine-O-sulphonic acid to obtain compound (V)

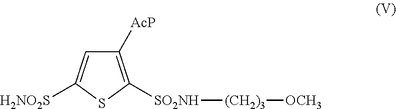

wherein AcP is as described above,
(d) (ii) reconstituting the acetyl group, e.g. by deprotecting or oxidizing compound (V) to obtain compound (Va):

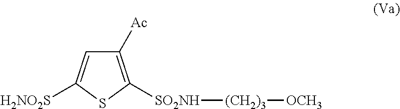

wherein Ac is an acetyl group,
(e) reacting compound (Va) with a brominating agent, particularly pyridinium bromide perbromide (PBP) to obtain compound (VI)

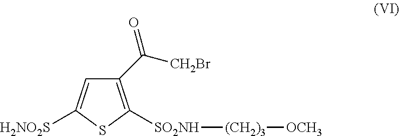

(f) reacting compound (VI) with a reducing agent, particularly a chiral reducing agent, more particularly (+)-diisopinocamphenylchloro-borane (DIPCl) under alkaline conditions to obtain compound (VII)

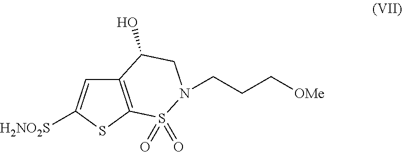

(g) (i) converting compound (VII) to the intermediate (VIIa),

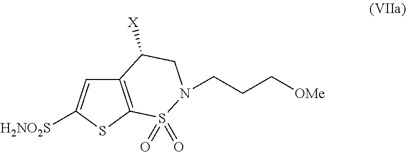

wherein X is a leaving group, particularly a sulfonate group, and
(g) (ii) reacting compound (VIIa) with ethylamine to obtain compound (VIII)

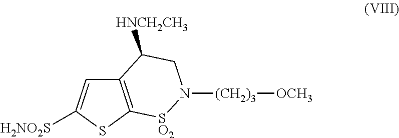

(h) optionally purifying compound (VIII), e.g. with a chiral tartaric acid, with water and/or with alcohol or aqueous alcohol, particularly iso propanol, to remove undesired isomers along with other impurities.

Step (a) comprises the reaction of 3-acetyl-2,5-dichlorothiophene or a protected acetyl derivative thereof, i.e. compond (I) with a sulfonating agent, particularly a sulphite salt, e.g. an alkali metal sulphite such as sodium sulphite in a suitable solvent, e.g. ethanol/H₂O under suitable reaction conditions, e.g. 10-20 h under reflux. The product is obtained preferably in a yield of greater than 90%.

Step (b) comprises reacting compound (II) with 3-methoxy amino propane. For this reaction step, compound (II) is first contacted with phosphorous pentachloride (Pcl₅), thionyl chloride, sulfuryl chloride, or phosphorus oxytrichloride under water-free conditions. Then, after neutralisation, e.g. with sodium bicarbonate, methoxypropylamine is added to obtain compound (III), i.e. 3-acetyl-5-chloro-thiophene-2-sulfonyl-N-(3-methoxypropyl) amide or an acetyl protected derivative thereof. The reaction may be carried out in an aqueous solvent, e.g. under cooling for about 1 to about 5 h. The product is preferably obtained in a yield of greater than 50%.

Step (c) comprises masking the acetyl group of compound (III). In a first process variant, this step involves reacting compound (III) with an acetyl protecting agent, if an unprotected acetyl group is present. Preferably, the acetyl protecting agent is ethylene glycol which reacts with compound (III) in the presence of sulphonic acids such as p-toluene sulfonic acid or camphor sulfonic acid, Lewis acids such as borontrifluoride etherate, or acidic resins such as Amberlite IR120, or Nafion H, under heating for e.g. 8-15 h. The acetyl protected compound (IV) is preferably obtained in a yield of 80% or higher.

In a second process variant, this step involves reacting compound (III) with a reducing agent, e.g. a borane-reducing agent such as NaBH₄ in a suitable solvent, e.g. an alcohol, under cooling, e.g. at 5-10° C. for e.g. 0.5-2 h. Thereby, the acetyl group is reduced to a 1-hydroxy-ethyl group. The reduced compound (IV) is preferably obtained in a yield of 80% or higher.

Step (d)(i) comprises reacting protected or reduced compound (IV) with an organo metal (organometallic) compound, preferably an organo lithium compound, more preferably an alkyl lithium compound such as butyl lithium, followed by treatment with SO₂ and subsequently with hydroxylamine-O-sulphonic acid. The reaction with the organo lithium and sulphurdioxide is preferably carried out under cooling, e.g. below −70° C. in an anhydrous solvent e.g. tetrahydrofuran under a protective atmosphere. The subsequent reaction with hydroxylaminde-O-sulphonic acid may be carried out in an aqueous solvent. The yield of compound (V) is preferably 60% or higher.

Step (d)(ii) comprises reconstituting the acetyl group. In the first process variant, this step involves the removal of the acetyl protecting group in order to obtain compound (Va). Preferably, the acetyl group is removed under acidic conditions, e.g. by adding HCl in a suitable solvent such as acetonitrile/water. The yield of compound (Va) is preferably 80% or higher.

In the second process variant, this step involves oxidizing the 1-hydroxy-ethyl group with a suitable oxidizing agent, e.g. the Dess-Martin periodinane (1,1,1-triacetoxy-1,1-dihydro-1,2-benziodoxol-3[1H]one) in a suitable solvent, e.g. dichloromethane, under light cooling, e.g. at 10-20° C. for a suitable time period, e.g. 0.5-4 h. The yield of compound (Va) is preferably 60% or higher.

Step (e) comprises reacting compound (Va) with a suitable brominating agent, particularly pyridinium bromide perbromide (PBP) preferably under acidic conditions, e.g. in the presence of sulfuric acid in a suitable solvent such as ethyl acetate. The yield of compound (VI) is preferably 80% or higher.

Step (f) comprises reacting compound (VI) with a reducing agent under alkaline conditions. Preferably, the reducing agent is a chiral reducing agent which gives in a stereoselective reduction compound (VII), the R-enantiomer. More preferably, the reducing agent is a chiral organic borane compound such as (+)-diisopinocamphenylchloroborane. Alternatively, step (f) may also comprise a reaction of compound (VI) with a non-chiral reducing agent such as NaBH₄. The use of a non-chiral reducing agent, however, requires greater efforts in order to remove undesired isomers, e.g. the (S) isomer of brinzolamide and is thus less preferred. The reduction is preferably carried out under anhydrous conditions in a suitable solvent, e.g. t-butylmethylether under a protective atmosphere. Compound (VII) is preferably obtained in a yield of 60% or higher.

Step (g)(i) comprises converting compound (VII) to the intermediate (VIIa), e.g. by reaction with a suitable acid anhydride, such as methanesulfonic anhydride in an organic solvent such as tetrahydrofuran and preferably under cooling. From this reaction mixture brinzolamide, i.e. compound (VIII) is preferably obtained without any work-up by reaction with ethylamine, preferably in an aqueous solvent. The yield of crude brinzolamide (VIII) is preferably 60% or higher.

Optional step (h) comprises purification or brinzolamide to remove impurities, particularly undesired isomers. Preferably, purification may comprise treatment with a chiral tartaric acid, treatment with water at elevated temperature and/or recrystallization from alcohol or aqueous alcohol, e.g. isopropyl alcohol as described in detail below. The yield of pure brinzolamide from step (b) is preferably 80% or higher.

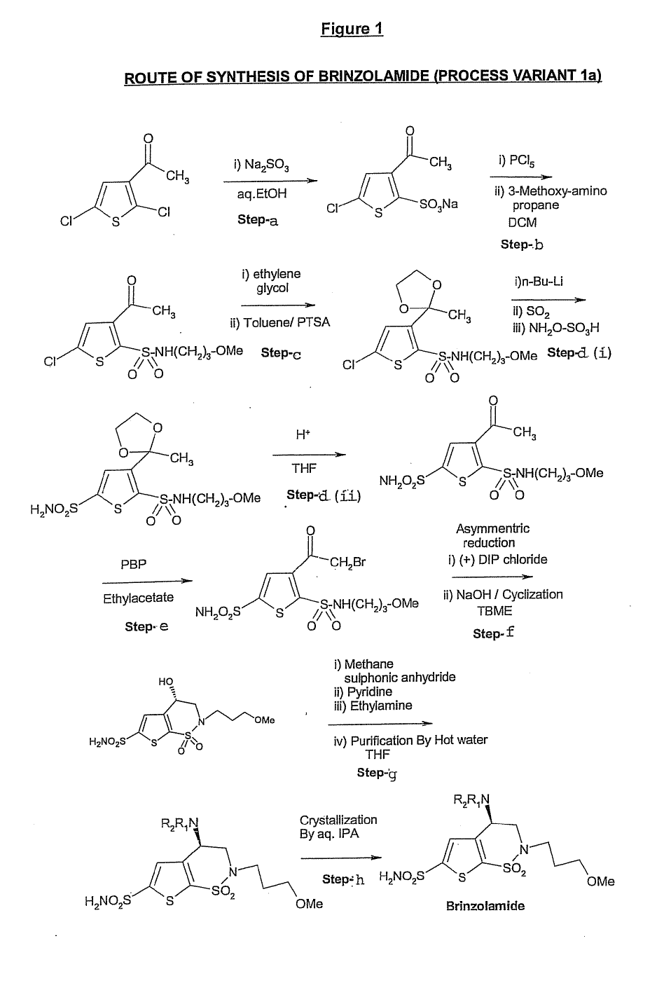

FIG. 1 shows a preferred embodiment of the inventive process (variant I a) using a chiral reducing agent in order to carry out an asymmetric reduction in step (f).

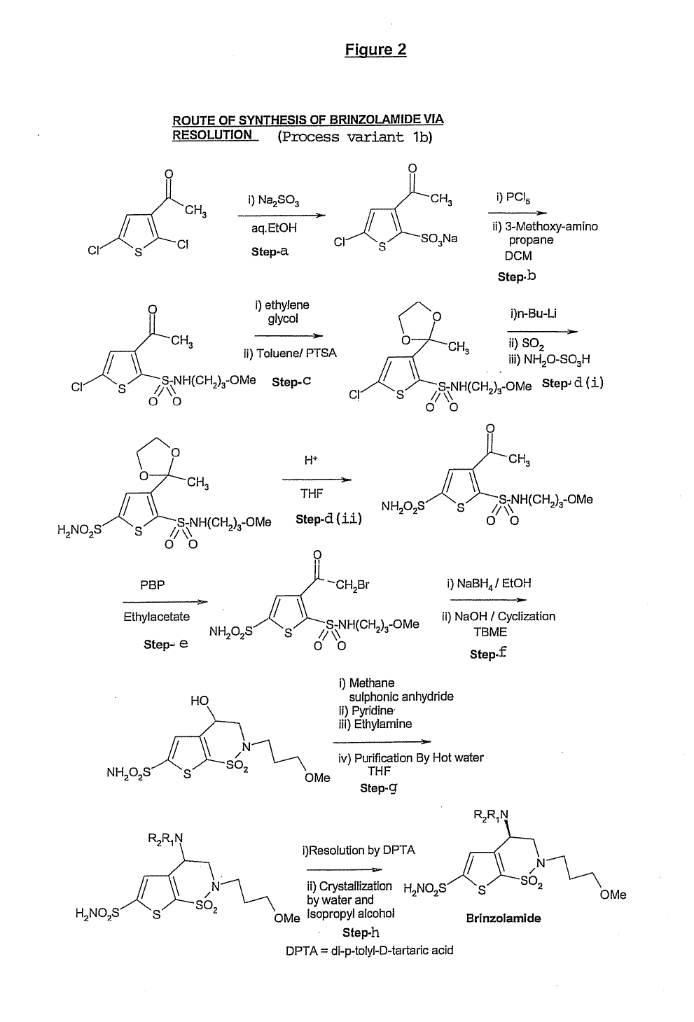

FIG. 2 shows a further embodiment of the inventive process (variant 1 b) comprising the use of a non-chiral reducing agent in step (f) and subsequent optical resolution, e.g. by using di-p-tolyl-D-tartaric acid (DTPA).

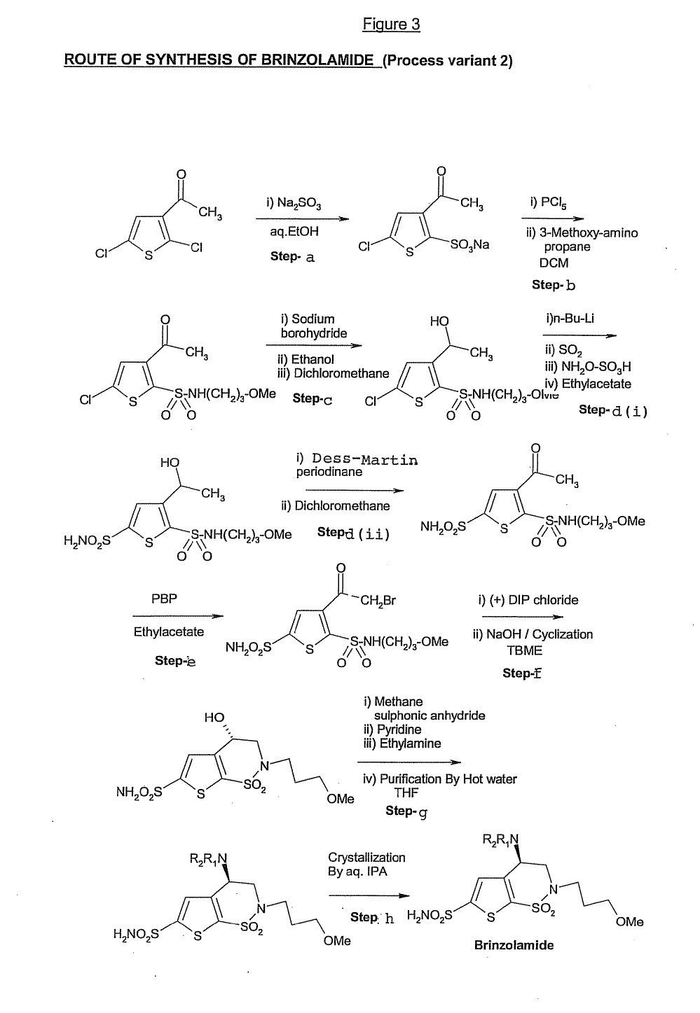

FIG. 3 shows a further preferred embodiment of the inventive process (variant 2) comprising a reduction/oxidation step of the acetyl group.

The present invention presents clear advantages in view of known methods, as it employs a cost effective process with significantly higher yields for preparation of the stage II material. Moreover, the above process is operationally more simple and safe as compared to the methods documented in literature.

The process also presents significant advantages with respect to preparation of compound V, step d (i) of process variant (2) as the lithiation proceeds at −40 to −20° C. with a shorter reaction time and also does not lead to the formation of any ortho lithiated compound as has been reported in literature for lithiation with the cyclised intermediate (Ref: Org. Proc. Res. & Dev., 1999, 3, 114-120).

Further, the present invention refers to a novel compound having the structural formula (II):

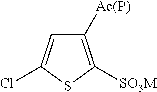

wherein Ac(P) is an acetyl group or a masked, e.g. protected or reduced acetyl group and M is a metal cation, particularly Na⁺.

Furthermore, the present invention refers to a compound having the structural formula (V):

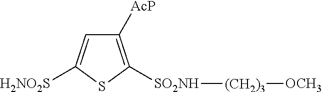

wherein AcP is a masked, e.g. protected or reduced acetyl group, particularly

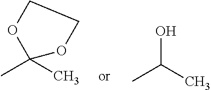

Furthermore, the present invention refers to a compound having the structural formula (VI):

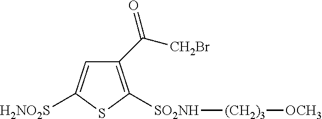

Furthermore, the present invention refers to a compound having the structural formula of formula (VII):

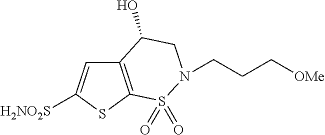

Still a further subject-matter of the present invention is the use of compounds (II), (V), (VI) and/or (VII) for the preparation of brinzolamide, particularly in a process as described above.

Still a further subject-matter of the present invention is a process for preparing brinzolamide, wherein at least one compound selected from compounds (II), (V), (VI) and (VII) is obtained as an intermediate.

The present invention also refers to novel method for purifying brinzolamide. A first aspect in this embodiment is a process for purifying brinzolamide from impurities, particularly from the S-enantiomer comprising forming a solution comprising a crude brinzolamide and a chiral tartaric acid, precipitating the desired brinzolamide tartrate from the solution and recovering the purified brinzolamide product.

The chiral tartaric acid is preferably di-p-tolyl-D-tartaric acid (DTPA). The process preferably comprises contacting the crude brinzolamide starting material (comprising R and S isomer) and the chiral tartaric acid to form a solution, e.g. by heating in a suitable solvent, e.g. aqueous methanol. Subsequently, the solution temperature is lowered, e.g. to room temperature and kept there for sufficient time, e.g. 8-24 h or longer to allow precipitation of the desired R-brinzolamide tartrate from the solution. The precipitated tartrate salt is isolated from the solution. Subsequently, the isolated tartrate salt may be neutralised, e.g. with bicarbonate, to obtain the desired free base of brinzolamide. The purified brinzolamide base has preferably a content of undesired S-enantiomer of 0.50 wt-% or less. If the content of undesired S-ennatiomer is higher, additional purification processes as described below in detail may have to be performed.

This embodiment of the present invention also refers to a salt of brinzolamide with a chiral tartaric acid, particularly a salt of brinzolamide with DTPA. Preferably, this brinzolamide salt has a content of undesired S-enantiomer of less than 50 wt-%.

Still a further aspect of the present invention refers to a process for purifying brinzolamide from impurities, particularly from the S-enantiomer comprising treating a crude brinzolamide with water at elevated temperature and recovering the purified brinzolamide product.

Surprisingly, it was found that suspending a crude brinzolamide product in water at an elevated temperature, e.g. from 60-80° C., preferably from 65-70° C. for a suitable time, e.g. for 30 min to 2 h, preferably from 45 min to 1 h, subsequent cooling to about 40-50° C. and filtering the contents at these conditions leads to a substantial reduction of the amount of undesired S-enantiomer. Preferably, the content of undesired S-enantiomer in the resulting purified brinzolamide product is 0.50 wt-% or less. If necessary, the water purification process may be repeated once or several times, e.g. up to 5 times.

Still a further aspect of the present invention is a process for purifying brinzolamide from impurities, particularly from the S-enantiomer comprising recrystallising a crude brinzolamide in isopropyl alcohol and recovering the purified brinzolamide product.

Surprisingly, it was found that recrystallisation of crude brinzolamide from isopropyl alcohol leads to a substantial reduction of the content of undesired S-enantiomer. Preferably, the content of undesired S-enantiomer in the purifled brizolamide product is 0.50 wt-% or less. If necessary, the isopropyl recrystallisation procedure is repeated, e.g. up to 4 times.

In an especially preferred embodiment the purification of crude brinzolamide comprises a combination of at least two of the following procedures:

(i) optical purification with a chiral tartaric acid,

(ii) treatment with water at elevated temperature,

(iii) recrystallisation in isopropyl alcohol.

Especially preferred is a treatment in water at elevated temperature in combination with isopropyl alcohol recrystallization.

Furthermore, the invention is described in greater detail by the following Examples.

### EXAMPLE 1

### Preparation of Brinzolamide (VIII) (Process Variant 1)

Step (a): Preparation of Sodium Salt (II)

3-Acetyl-2,5-dichlorothiophene (I) (500 g, 2.56 mol) was suspended in ethanol (2.50 L). Sodium sulphite solution (5.0 L, 12.8 mol) was added slowly at 25-30° C. and heated to reflux at 100-110° C. for 16 h. The completion of the reaction was monitored by HPLC [(HPLC limit of 3-acetyl-2, 5-dichlorothiophene: NMT (not more than) 1.0%); Observed: 0.1% of 3-acetyl-2,5-dichlorothiophene].

The reaction mixture was cooled to 25-30° C., filtered and washed with anhydrous methanol (1.0 L). The residue obtained was kept aside and the filtrate concentrated under reduced pressure. The resulting crude product was suspended in methanol (5 vol), stirred for 1.0 h, filtered and washed with anhydrous methanol (1 vol). The filtrate was concentrated under reduced pressure, dried under vacuum at 60-65° C., until a constant weight was obtained.

Yield of Compound (II): 640 g (95.5%)

Step (b): Preparation of N-Substituted Sulfonamide (III)

To a stirred suspension of compound (II) (640 g, 2.44 mol) in dichloromethane (6.50 L; moisture content NMT 0.5%) at 10-20° C., phosphorus pentachloride solution (PCl₅) (7.20 moles, 1.575 kg in 12.80 L of dichloromethane) was added slowly dropwise over a period of 1-2 h and stirred for 4.0-5.0 h at 10-20° C. under N₂ atmosphere. The completion of the reaction was monitored by HPLC analysis [HPLC limit of sodium salt: NMT 1.0%].

The reaction mixture was cooled, filtered and the filtrate quenched with saturated sodium bicarbonate solution (5.0 L) (to remove excess PCl₅ traces until the aqueous layer pH was 7). The filtrate was washed, separated and dried over sodium sulphate and filtered. The filtrate was stirred at 5-10° C., thereafter methoxypropylamine (435 g, 4.88 mol) was added slowly and stirred for 2 h at 5-10° C. The completion of the reaction was monitored by HPLC [(HPLC limit of 3-acetyl-5-chlorothiophene-2-sulfonyl chloride: NMT 1.0%; Observed: 0.10% of 3-acetyl-5-chlorothiophene-2-sulfonyl chloride]. The reaction mixture was washed with purified water (6×1.0 vol) with continuous stirring. After 2 water washings (2×1.0 vol) the pH of the solution was adjusted to pH 8 with triethylamine. The remaining water washings (4×1.0 vol) were conducted at a pH of 8.

The organic layer was separated and dried over sodium sulphate, and was distilled off completely. The product obtained was washed with t-butylmethyl ether (3×1.0 vol) followed by hexane (3×1.0 vol), and dried at 35-40° C. until constant weight was obtained.

Yield of Compound (III): 400 g (52.6%)

Step (c): Preparation of Protected N-Substituted Sulfonamide (IV)

To a suspension of compound (III) (400 g, 1.285 mol) in dry toluene (20 vol) at 25-30° C. ethylene glycol (360 ml, 6.425 mol) and p-toluene-sulfonic acid (120.0 g, 0.642 mol) were added. The reaction mixture was stirred at 75-80° C. for 10.0-12.0 h. The completion of the reaction was monitored by HPLC [(HPLC limit of N-substituted sulfonamide: NMT 1.0%; in mobile phase ethyl acetate:hexane, 1:9)].

The reaction mixture was cooled to 25-30° C. and the organic layer separated, washed with saturated sodium bicarbonate solution (2×2.0 L) and chilled water (2×2.0 L). The organic layer was separated, dried over sodium sulphate, filtered and distilled completely under reduced pressure. The crude product obtained was a pale yellow colored syrupy liquid.

Yield of Compound (IV): 373 g (81.7%)

Step (d)(i): Preparation of Protected 5-sulphonamide (V)

A solution of compound (IV) (373 g, 1.046 mol) in anhydrous tetrahydrofuran (THF) (15 vol) was cooled to −70° C. under nitrogen. n-Butyl lithium (1.28 L, 1.404 moles, 2.5M solution) was added drop wise over 2.5 h while the temperature was kept below −70° C. The reaction mixture was maintained for 1.5-2.0 h below −70° C., after which sulphurdioxide (SO₂) was introduced into the reaction mixture until an aliquot quenched into water showed a pH 4.0. The mixture was allowed to warm at 25-30° C. for 15 hrs and then concentrated.

The residue was dissolved in water (2.5 vol) and the solution was added in one portion to a precooled solution of sodium acetate trihydrate (1.175 Kg, 6.265 mol) and hydroxylamine-O-sulfonic acid (615 g, 4.202 mol) in water (3.0 vol), causing the temperature to rise to 25° C. After being stirred for 15 hrs at 25-30° C., the solution was neutralized with sodium bicarbonate and then basified with 50% sodium hydroxide solution until pH 9.0 was attained. The solution was extracted with ethyl acetate, washed with brine, dried with sodium sulphate, filtered and concentrated. The organic layer was distilled completely below 50° C. under reduced pressure. The crude product obtained was a pale yellow colored semi-solid.

Yield of Compound (V): 280 g (66.6%)

Step (d)(ii): Preparation of Deprotected 5-sulphonamide (Va)

To a solution of compound (V) (280 g, 0.700 moles) in acetonitrile (5 vol) 2N HCl (5 vol) was added. The mixture was refluxed at 75-80° C. for 4.0 h. The reaction was monitored by TLC [TLC mobile phase ethyl acetate:hexane, 1:1].

After the reaction, the acetonitrile was removed completely under vacuum and the reaction mixture basified with saturated sodium bicarbonate solution to pH 8.0.The aqueous layer was extracted with ethyl acetate (3×3.0 vol), dried over sodium sulphate and filtered. The organic layer was distilled completely below 50° C. under reduced pressure. The crude product obtained was a pale yellow colored syrupy liquid.

Yield of Compound (Va): 210 g (84.2%)

Step (e): Preparation of Bromo Compound (VI)

Compound (Va) (210 g, 0.588 moles) was suspended in ethyl acetate (10 vol) and stirred. The pale yellow suspension was cooled to 0-5° C. over 30 minutes and pyridinium bromide perbromide (188 g, 0.588 mol) was added in one portion. To the above suspension, sulfuric acid (0.5 vol) was added via an addition funnel over 15-25 minutes causing the temperature to rise to 5° C. After the addition of sulfuric acid, the reaction mixture was gradually bought to 25-30° C. and stirred for one hour at this temperature. The reaction was monitored by TLC [mobile phase ethyl acetate:hexane, 3:7].

After completion of the reaction, ice cooled water was added to the reaction mixture. The organic layer was separated, washed with water, brine, dried over sodium sulphate, filtered and the solvent removed in vacuum (below 50° C.) to yield the crude product as a yellow syrup.

Yield of Compound (VI): 210 g (82.00%)

Step (f): Preparation of Cyclised Product (VII)

The cyclisation of compound (VI) may be carried out using a chiral reducing agent such as (+)-diisopinocamphenylborane (DIPCl) in order to obtain the desired isomer of compound (VII) by a stereoselective reduction.

Compound (VI) (210 g, 0.484 moles) was suspended in t-butyl methyl ether (15 vol) and stirred under nitrogen atmosphere. The stirred suspension was cooled to −40° C. and (+)-diisopinocamphenylborane (DIPCl) (762 g (60-65% in hexane solution), 1.063 moles) was added via a cannula over a period of 30 minutes. A rise in temperature (up to −30° C.) was observed. The reaction mixture was maintained at −25 to -20° C. for 3.0 to 4.0 h and monitored by TLC [mobile phase ethyl acetate:hexane, 3:7].

After completion of the reduction, 1M aqueous sodium hydroxide solution (20 vol) was added from an addition funnel over a period of 15-20 minutes, The reaction mixture was stirred at 25-30° C. overnight and monitored by TLC [Mobile phase Ethyl acetate:hexane, 1:1] for completion. After completion, the aqueous and organic phases were separated and the aqueous phase was extracted with t-butyl methyl ether (4.0 vol), acidified to pH 1 using 5N hydrochloric acid solution and extracted with ethyl acetate (3×6.0 vol). The combined ethyl acetate extracts were washed with saturated brine solution (2×2.5 vol), dried over sodium sulphate and filtered. The organic layer was distilled completely (below 50° C.) under reduced pressure. The crude product obtained was a pale yellow colored syrup.

Yield of Compound (VII): 112 g (65.2%)

Alternatively, step (f) may also be carried out using a non-chiral reducing agent such as sodium tetrahydroborane. In this case, subsequent optical resolution steps, i.e. removal of undesired isomer, have to be carried out.

Step (g): Preparation of Crude Brinzolamide (VIII)

Compound (VII) (112 g, 0.313 mol) was suspended in tetrahydrofuran (10 vol). Methanesulfonic anhydride (65.7 g, 0.374 moles) was added in one lot and stirred. The reaction mixture was cooled to 0-5° C. and pyridine (2.5 vol) was added slowly over a period of 45 minutes. The reaction was monitored by TLC [Mobile phase dichloromethane:methanol, 9.5:0.5] for completion.

As reaction product the intermediate compound (VIIa) was obtained.

To the above reaction mixture 70% aqueous ethylamine solution (400 ml, 10 vol) was added slowly at 0-5° C. After the complete addition, the reaction mixture was stirred at 25-30° C. for 12.0 h. The reaction is monitored by TLC [Mobile phase dichloromethane:methanol, 9.5:0.5] for completion. The organic layer (ethylamine and tetrahydrofuran mixture) was distilled off completely below 50° C. under reduced pressure. The aqueous phase was acidified with concentrated hydrochloric acid and the pH adjusted to 3.0 at 15-25° C. The aqueous phase was extracted with t-butyl methyl ether twice (2×2.0 vol) to remove any organic impurities if present. The t-butyl methyl ether layer was extracted with 1N HCl solution and the layers separated.

The aqueous layers were combined, treated with activated charcoal and heated at 65-70° C. The contents were stirred at 65-70° C. for 30 minutes, filtered through hyflo supercel at 65-70° C. and washed with preheated water (65-70° C.). The filtrate was cooled to 25-30° C. and the pH was adjusted to 8 with solid sodium bicarbonate. The reaction mixture was stirred for 12 h for crystallization. If the crystallization did not occur, the reaction mass was filtered through a Büchner funnel, extracted with ethyl acetate (3×5.0 vol), dried over sodium sulphate and filtered. The organic layer was distilled completely (below 50° C.) under reduced pressure. The crude product was obtained as a pale yellow syrup, which was washed with hexane (4×1.0 vol) to yield a residue which crystallized to a solid on standing.

Yield of Crude Compound (VIII): 80 g (66.3%)

Step (h): Preparation of Pure Brinzolamide (VIII)

Crude compound (VIII) (80.0 g, 0.208 moles) was suspended in water (5.0 vol) and heated to 65-70° C. for one hour, filtered (in hot) and sucked dry for 30 minutes. The process was repeated by chiral, till the other isomer content was NMT: 0.50%.

The product thus obtained was suspended in isopropyl alcohol (8.0 vol) and heated to 65-70° C., maintained for 30 minutes and filtered under hot condition. To the dark solution, activated carbon (10.0% by weight) was added and heated at 65-70° C. for 30 minutes. The solution was filtered under hot conditions and washed with isopropyl alcohol (preheated at 65-70° C.). The solution was transferred to another flask and stirred for one hour at 25-30° C. A crystalline solid precipitate was filtered, sucked dry for 30 minutes and dried under vacuum at 65-70° C., until a constant weight obtained.

Yield of Pure Compound (VIII): 60 g (86%)

### EXAMPLE 2

Preparation of Brinzolamide (Process Variant 2)

Step (a): Preparation of Sodium Salt (H)

3-Acetyl-2,5-dichlorothiophene (I) (500 g, 2.56 mol) was suspended in ethanol (2.50 L).Sodium sulphite solution (obtained by dissolving sodium sulphite in water i.e. 1.61 Kg in 5.0 L of water, 12.8 moles) was added slowly at 25-30° C. and heated to reflux at 100-110° C. The reaction mixture was refluxed for 16 hrs at 100-110° C. The completion of the reaction was monitored by HPLC [(HPLC limit of 3-acetyl-2,5-dichlorothiophene: NMT 1.0%); Observed: 0.1% of 3-acetyl-2,5-dichlorothiophene].

The reaction mixture was cooled to 25-30° C., filtered and washed with anhydrous methanol (1.0 L). The filtrate was concentrated (at 60-65° C.) under reduced pressure. The resulting crude product was suspended in anhydrous methanol (6.0 L) and stirred for 30 min at 25-30° C. The undissolved solid was filtered and the filtrate was concentrated at 60-65° C. completely under reduced pressure. The material was dried under vacuum at 60-65° C., until constant weight is obtained.

Yield: 640 g of Compound (II) (95.5%)

Step (b): Preparation of N-Substituted Sulfonamide (III)

To a stirred suspension of compound (II) (640 g, 2.44 mol) in dichloromethane (6.50 L; moisture content NMT 0.5%), phosphorus pentachloride (PCl₅) solution (1.575 Kg in 12.80 L of dichloromethane,) was added slowly over a period of 1-2 hrs time intervals at 10-20° C. The reaction was stirred for 4.0 h under N₂, while keeping the temperature The completion of the reaction was monitored by HPLC analysis [HPLC limit of Sodium salt: NMT 1.0%].

The cooled reaction mixture was filtered (to remove undissolved salts) and the filtrate was quenched with saturated sodium bicarbonate solution (5.0 L) (to remove excess PCl₅ traces until the aqueous layer pH was 7), and washed with water. The organic layer was separated, dried over sodium sulphate and filtered. To the stirred organic layer, methoxypropylamine (435 g, 4.88 mol) was added slowly and the reaction mixture stirred for 2.0 hrs at 5-10° C. The completion of the reaction was monitored by HPLC [(limit for 3-acetyl-5-chlorothiophene-2-sulfonyl chloride: NMT 1.0%); Observed: 0.1% of 3-acetyl-5-chlorothiophene-2-sulfonyl chloride]. The reaction was washed with 1N HCl solution (2 vol) and water. The organic layer was separated, dried over sodium sulphate, filtered and concentrated to yield the product. The product obtained was washed with hexane (2.0 L), and dried at 45-50° C. until a constant weight was obtained.

Yield of Compound (III): 350 g (46.2%)

Step (c): Preparation of Reduced Sulfonamide (IV)

To a solution of compound (III) (25 g, 0.080 mol) in absolute ethanol (15 vol), sodium borohydride (1.2 g, 0.032 moles), was added at 5-10° C. The reaction mixture was stirred at 5-10° C. for 45 min to one hour. The completion of the reaction was monitored by TLC [(TLC limit of N-substituted sulfonamide: NMT 1.0%; in mobile phase ethylacetate:hexane, 3:7)].

After the completion of the reaction, ethanol was removed and the residue was taken up in dichloromethane (5 vol). The dichloromethane solution was washed with water and brine (3 vol). The organic layer was separated, dried over sodium sulphate, filtered and distilled below 50° C. under reduced pressure. The crude product was obtained as a pale yellow syrup.

Yield of Compound (IV): 21.0 g (83.0%)

Step (d)(i): Preparation of Reduced 5-sulphonamide (V)

A solution of compound (IV) (20 g, 0.063 mol) in anhydrous tetrahydrofuran (15 vol) was cooled to −40° C. using dry ice/2-propanol under N₂. n-Butyl lithium (79 ml, 0.190 mol) was added dropwise over 30 min to one hour while the temperature was kept below −20° C. during the addition. The reaction mixture was maintained for 30 min to one hour below −40° C. The sulphurdioxide (SO₂) was introduced into the reaction mixture until an aliquot quenched into water gave a pH 3.0 to 4.0. The mixture was allowed to warm at 25-30° C. for 15 h and then concentrated.

The residue was dissolved in water (100 ml) and to this solution was added in one portion (at 0° C.), a solution of sodium acetate trihydrate (31.5 g, 0.38 mol) and hydroxylamine-O-sulfonic acid (29 g, 0.252 mol) in water (0.3 L), causing the temperature to rise to 25° C. After being stirred for 15 h at 25-30° C., the solution was neutralized with sodium bicarbonate and then basified with 50% sodium hydroxide solution until a pH 8-9 was attained. The solution was extracted with ethyl acetate, washed with brine, dried with sodium sulphate, filtered and then concentrated. The organic layer was distilled completely below 50° C. under reduced pressure. The crude product was obtained as a pale yellow syrup which was crystallised with DCM/hexane to yield the compound as a pale yellow solid.

Yield of Compound (V): 16.06 g (70%)

Step (d)(ii): Preparation of 5-sulphonamide (Va)

To a suspension of compound (V) (10 g, 0.028 mol) in dichloromethane(15 vol) suspension of Dess-Martin periodinane (11.80 g, 0.028 mol in 5 vol of dichloromethane) was added slowly at 10-20° C. for 1.0 to 2.0 h. The reaction was monitored by TLC [TLC limit of reduced 5-sulphonamide: NMT 1.0%; in mobile phase dichloromethane:methanol, 9.5:0.5)].

After the completion of the reaction, the reaction mixture was quenched with purified water (20 vol) and stirred for 45 minutes to 1.0 hr. The organic layer was separated and washed with saturated sodium bicarbonate solution (2×5.0 vol) and sodium thiosulphate solution (10%), dried over sodium sulphate, filtered and distilled completely below 50° C. under reduced pressure. The resulting crude product was a pale yellow syrup. The crude product was crystallized from dichloromethane and hexane to yield the pure compound as a pale yellow/light brown crystalline solid.

Yield of Compound (Va): 6 g (65%)

The further steps were carried out as described in Example 1.

### EXAMPLE 3

Process for the Optical Resolution of Brinzolamide

Crude brinzolamide (0.065 mol) was dissolved in 5.0% aq. methanol (4.0 vol) and heated for one hour at 50° C. Simultaneously, a solution di-p-tolyl-D-tartaric acid (DPTA) dissolved in methanol (1.0 vol) was prepared and stirred at 25-30° C. for 20-30 minutes. The DPTA solution was added slowly to the solution containing crude brinzolamide at 50° C. while stirring. The mixture was maintained for 1.0 h at 50° C. After one hour, the solution was slowly bought to 25-30° C. and continuously stirred for 12 h at 25-30° C. The solid that precipitates out was filtered, washed with cold methanol (1.0 vol) and dried to give the tartrate salt.

The tartrate salt was mixed with saturated sodium bicarbonate solution (60 vol) and the resulting suspension was stirred for 1.0 h and then was extracted with ethyl acetate (3×60 vol). The extracts were dried over sodium sulphate, filtered and evaporated to dryness to obtain the free base of brinzolamide. The free base was checked for the S-isomer content by chiral HPLC, Limit NMT: 0.50% (S-isomer).

If the content of the undesired S-isomer is higher than 0.50%, a water purification process and/or recrystallisation from isopropanol as described below may be performed to reduce the undesired isomer.

### EXAMPLE 4

a) Water Purification Process

Brinzolamide base was suspended in purified water (10 vol) and heated to 65-70° C. for 45 min-1.0 h. The mixture was allowed to cool to 50° C. and the contents were filtered under hot conditions, washed with purified water (2.0 vol) and sucked dry for 1.0 h. The process may be repeated (e.g. 4-5 times) till the limit of S-isomer content NMT 0.50% is obtained. Up to 10.00-20.00% of S-isomer content can be reduced to a limit of NMT 0.50% by repeated water purification.

b) Recrystallisation from Isopropanol

Crude brinzolamide (80.0 g, 0.208 mol) was suspended in isopropyl alcohol (8.0 vol) and heated to 65-70° C., maintained for 30 minutes and filtered under hot conditions. To the dark solution, activated carbon (10.0% by weight) was added and heated at 65-70° C. for 30 minutes. The solution was filtered under hot conditions and washed with isopropyl alcohol (preheated at 65-70° C.). The solution was transferred to another flask and stirred for one hour at 25-30° C. A crystalline solid precipitated. The precipitate was filtered, sucked dry for 30 minutes and dried under vacuum at 65-70° C., until a constant weight was obtained. The process may be repeated, until the product comprises ≦0.50% of the undesired isomer.

Yield: 60 g of Pure Brinzolamide (VIII) (86%)

1. A process for preparing brinzolamide comprising the steps:
(a) reacting compound (I) 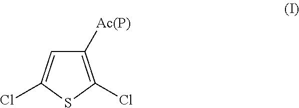
wherein Ac(P) is an acetyl group or a masked, e.g. protected or reduced acetyl group, with a sulphonating reagent, particularly a sulphite salt, to obtain compound (II) 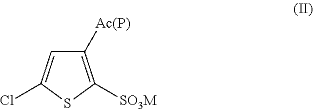
wherein Ac(P) is as described above and M is a cation, particularly Na⁺,
(b) reacting compound (II) with 3-methoxy amino propane to obtain compound (III) 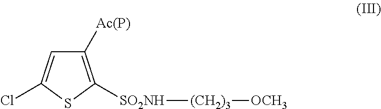
wherein Ac(P) is as described above,
(c) optionally reacting compound (Ill) with an acetyl protecting agent, particularly ethylene glycol, to obtain compound (IV) 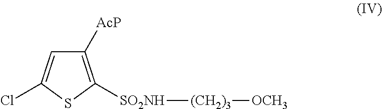
wherein AcP is a masked, e.g. protected or reduced acetyl group, particularly 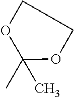
(d) (i) reacting compound (IV) with an organo metal compound followed by treatment with SO₂ and subsequently with hydroxylamine-O-sulphonic acid to obtain compound (V) 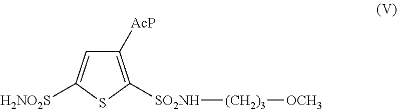
wherein AcP is as described above,
(d) (ii) reconstituting the acetyl group, e.g. by deprotecting or oxidizing compound (V) to obtain compound (Va): 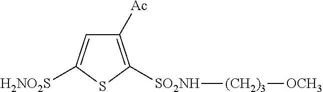
wherein Ac is an acetyl group,
(e) reacting compound (Va) with a brominating agent, particularly pyridinium bromide perbromide (PBP) to obtain compound (VI) 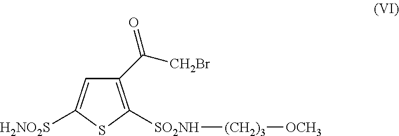
(f) reacting compound (VI) with a reducing agent, particularly a chiral reducing agent, more particularly (+)-diisopinocamphenylchloroborane (DIPCl) under alkaline conditions to obtain compound (VII) 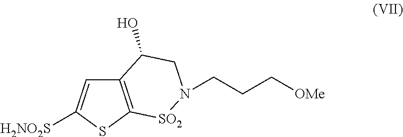
(g) (i) converting compound (VII) to the intermediate (VIIa), 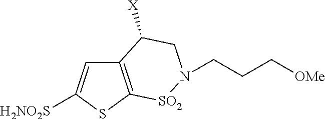
wherein X is a leaving group, particularly a sulfonate group, and
(g) (ii) reacting compound (VIIa) with ethylamine to obtain compound (VIII) 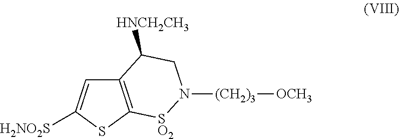
(h) optionally purifying compound (VIII) to remove undesired isomers along with other impurities.

2. A compound having the structural formula (II) 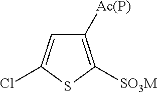
wherein Ac(P) is an acetyl group or a masked, e.g. protected or reduced acetyl group and M is a metal cation, particularly Na⁺.

3. A compound having the structural formula (V) 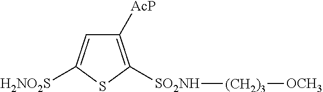
wherein AcP is a masked, e.g. protected or reduced acetyl group, particularly

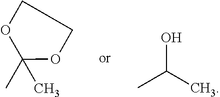

4. A compound having the structural formula (VI)

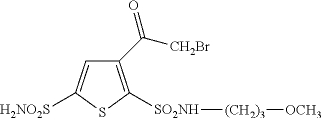

5. A compound having the structural formula (VII)

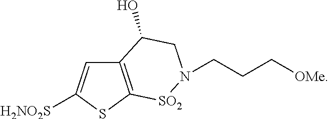

6. Use of a compound of any one of claims 2-5 for the preparation of brinzolamide.

7. A process for preparing brinzolamide wherein at least one compound of any one of claims 2-5 is obtained as an intermediate.

8. A process for purifying brinzolamide from impurities, particularly from the S-enantiomer, comprising forming a solution comprising a crude brinzolamide and a chiral tartaric acid, precipitating the desired brinzolamide tartrate from the solution and recovering the purified brinzolamide product.

9. The process of claim 8, wherein the chiral tartaric acid is di-p-tolyl-D-tartaric acid (DTPA).

10. The process of claim 8 or 9, wherein the content of undesired S-enantiomer in the purified brinzolamide product is 0.50 wt-% or less.

11. A salt of brinzolamide with a chiral tartaric acid, particularly a salt of brinzolamide with DTPA.

12. A process for purifying brinzolamide from impurities, particularly from the S-enantiomer, comprising treating a crude brinzolamide with water at elevated temperature and recovering the purified brinzolamide product.

13. A process for purifying brinzolamide from impurities, particularly from the S-enantiomer, comprising recrystallising a crude brinzolamide in isopropyl alcohol and recovering the purified brinzolamide product.

14. The process of claim 12 or 13, wherein the content of undesired S-enantiomer in the purified brinzolamide product is 0.50 wt-% or less.
